(19) United States
(12) Patent Application Publication
(10) Pub. No.: US 2011/0137084 A1
(43) Pub. Date: Jun. 9, 2011

(54) METHOD FOR PRODUCING PURE OR ENRICHED Q10 COENZYME

(76) Inventors: Volker Berl, Kehl (DE); Karin Schein, Ludwigshafen (DE); Frank Wetterich, Wachenheim (DE)

(21) Appl. No.: 11/722,455

(22) PCT Filed: Dec. 17, 2005
(86) PCT No.: PCT/EP2005/013626

§ 371 Date: Dec. 14, 2009

(30) Foreign Application Priority Data
Dec. 22, 2004 (DE) 102004063006.2

(51) Int. Cl. C07C 46/10

(52) U.S. Cl. 568/324

(57) ABSTRACT

The present invention relates to a method for isolating coenzyme Q₁₀ of formula (I)

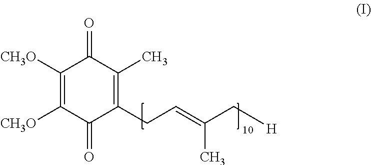

by separating material mixtures containing coenzyme Q₁₀ and the compound of formula (II)

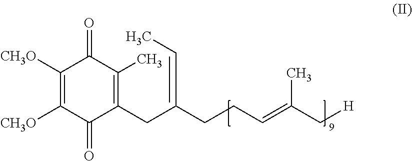

### TECHNICAL AREA OF THE INVENTION

The present invention relates to a method for producing pure or enriched coenzyme Q₁₀ by separating material mixtures containing coenzyme Q₁₀ and a constitutional isomer of coenzyme Q₁₀.

Coenzyme Q₁₀ (ubiquinone) of formula (I)

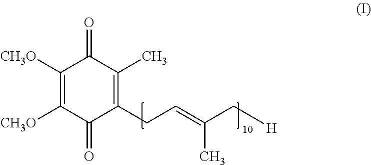

Is an important component of the human respiratory chain and has recently acquired increasing importance as a food supplement or therapeutic agent.

Totally synthetic approaches to coenzyme Q₁₀ often pursue a convergent strategy because of the size of the molecule. Accordingly, the aromatic or quinoid nucleus of the molecule and the polyisoprenoid side chain are usually firstly built up separately from one another and coupled to one another at a later stage of the synthesis.

### PRIOR ART

The coupling reaction may be carried out by a method described by Negishi et al. in Organic Letters, 2002, vol. 4, no. 2, 261-264, or, for the synthesis of coenzyme Q₆ or Q₇, by Lipshutz et al. in J. Am. Chem. Soc. 1999, 121, 11664-11673 by nickel-catalysed coupling of a vinylalane of formula (III)

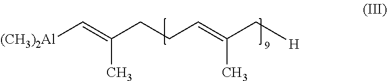

with a suitable quinone, for example one of the type of formula (IV)

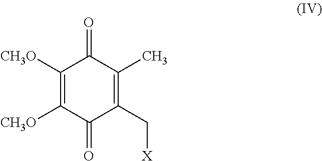

wherein X is a leaving group, such as, for example, halogen, especially chlorine.

The vinylalane to be used here of formula (III) is in turn accessible by carboalumination of the terminal alkyne of formula (V)

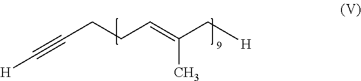

with trimethyl aluminium in the presence of a suitable catalyst, for example a zircon or titanium catalyst.

WO 2005/056812 discloses an improved method for producing ubiquinones, in particular coenzyme Q₁₀ by transition metal-catalysed coupling of a suitable quinone to an alkyne derivative of the respective ubiquinone side chain. The applicant further discloses mixtures of ubiquinones or ubiquinone derivatives with isomeric compounds, which have a constitutional isomeric side chain.

### OBJECT OF THE INVENTION

It has been shown that the carboalumination carried out in this manner does not exclusively lead to the desired carboalumination product of formula (III), but also to a regioisomeric vinylalane of formula (VI)

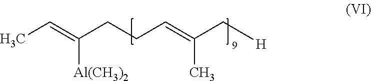

From the mixtures of the regioisomeric vinylalanes of formula (V) or (VI), by means of the aforementioned Ni-catalysed coupling, mixtures are obtained of coenzyme Q₁₀ of formula (I) and of the compound of formula (II)

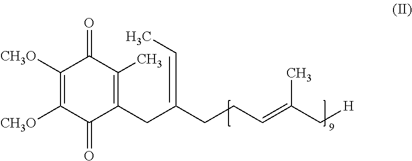

The present invention is based on the object of developing a method which allows mixtures of compounds of formula (I) and (II) to be treated in such a way that they are suitable for further applications, in particular for an application as a food supplement or therapeutic agent for humans.

### DESCRIPTION OF THE INVENTION AND PREFERRED EMBODIMENTS

The object was achieved according to the invention by providing a method for producing pure or enriched coenzyme Q₁₀ of formula (I)

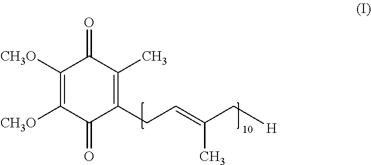

by separating material mixtures containing coenzyme Q₁₀ and the compound of formula (II)

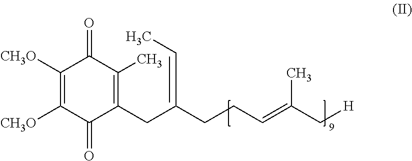

Said mixtures, as mentioned above may be obtained by Ni-catalysed coupling of a mixture of the isomeric vinylalanes of formulas (III) and (VI) with a suitable coupling partner, such as, for example, a quinone of formula (IV),

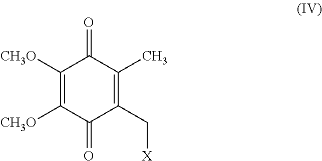

wherein X stands for a leaving group such as, for example, halogen, preferably chlorine or bromine, in particular chlorine or a radical OR, wherein R may signify, for example, hydrogen, a branched or unbranched alkyl radical with 1 to about 6 carbon atoms, such as, for example, methyl, ethyl, propyl, isopropyl, butyl, hexyl, cyclohexyl, or, together with the oxygen atom of the radical OR, sulphonyl such as methylsulphonyl, trifluoromethylsulphonyl, p-toluenesulphonyl and the like.

Said mixtures may contain further by-products, for example from previous synthesis stages of the leaving compounds. In particular, they may contain by-products or impurities, which occur in the production of alkyne of formula (V), for example by propargylation of solanesol derivatives, such as, for example, elimination products such as, for example, the compound of formula (VII)

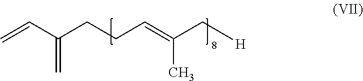

In addition, the material mixtures to be separated according to the invention may also contain, for example, reagents or catalysts, which are used in the carboalumination of the compound of formula (V) or the coupling of the vinylalanes of formulas (III) and (VI) obtained therefrom, such as, for example, Zr, Ti or Ni salts or else phosphines.

Preferred mixtures as starting materials for isolating coenzyme Q₁₀ by the method according to the invention are those in which coenzyme Q₁₀ is present, in addition to the compound of formula (II) or any impurities, as the main component in terms of weight, preferably at more than 30% by weight, in particular more than 40% by weight. Preferred mixtures as the starting material are in turn those which about 50% by weight, preferably more than about 80% by weight and in particular about 90 to about 99% by weight consist of coenzyme Q₁₀ and the isomeric compound of formula (II).

In said mixtures suitable as starting materials for isolating coenzyme Q₁₀, the molar ratio of coenzyme Q₁₀ to the isomer of formula (II) is advantageously about 85 to 15 up to about 99.7 to 0.3, preferably about 85 to 15 up to about 99.5 to 0.5, particularly preferably about 90 to 10 up to about 99.5 to 0.5, quite particularly preferably about 95 to 5 up to about 99.5 to 0.5.

The separation according to the invention can preferably be carried out by selective crystallisation of coenzyme Q₁₀ from solutions, which contain coenzyme Q₁₀ and the compound of formula (II). The term “selective” is taken to mean here that one of the two compounds of the formulas (I) or (II) is present in the crystallisate obtained in a more enriched form in comparison to the mixture used, i.e. that the molar ratio of said compounds in the crude product is shifted to the benefit of one of the two compounds in the crystallisate. The selective crystallisation or enrichment of coenzyme Q₁₀ of formula (I) is preferable in the crystallisate, in this case.

Preferred solvents for carrying out said selective crystallisation are alcohols, in particular those with 1 to about 10 carbon atoms such as, for example, methanol, ethanol, propanol, isopropanol, n-butanol, isobutanol, tert.-butanol, hexanol ethylene glycol, propanediol, butanediol and the like.

Further preferred solvents are carbonyl compounds, such as, for example, acetone, diethyl ketone, methyl ethyl ketone, acetic acid ethyl ester or cyclohexanone.

Mentioned as further preferred solvents are the cyclic or acyclic ethers such as, for example, diethyl ether, tetrahydrofurane, dioxane, methyl-tert.-butyl ether or diglyms.

Mentioned as further suitable solvents for carrying out the separation according to the invention are also halogenated solvents, such as, for example, dichloromethane or dichloroethane and aromatic solvents such as toluene or xylene.

Moreover, mentioned as suitable solvents are also hydrocarbons such as, for example, petrol ether, pentane, hexane, heptane, cyclohexane and the like.

Further solvents which are preferred in the scope of the present invention are acetonitrile and water.

Said solvents may also be used in the form of mixtures, in particular in the form of binary or ternary mixtures of said solvents. In the scope of the present invention, ethanol or solvent mixtures which contain ethanol are preferred as solvents. From amongst said solvent mixtures, preferred are those which contain ethanol as the main component in terms of weight, in particular those consisting more than about 70% by volume, preferably about 80 to about 100% by volume of ethanol. A particularly preferred solvent in the scope of the present invention is pure, i.e. at least about 95% by volume, ethanol.

In addition, the solvent mixtures preferred according to the invention are those which contain ethanol and/or acetone and water.

Depending on the solvent or solvent mixture selected, the concentration of the material mixture used in the solvent may be varied within broad limits. Such solutions which, based on the total solution, consist of about 1 to about 50% by weight, preferably from about 1 to about 35% by weight, particularly preferably from about 1 to about 10% by weight of said material mixtures containing coenzyme Q₁₀ and the compound of formula (II), are advantageously used to isolate coenzyme Q₁₀ by the separation method by means of crystallisation preferred according to the invention.

The preferred separation method according to the invention by crystallisation can be carried out at temperatures in the range from about −20° C. to about 80° C. preferably at about 0° C. to about 60° C., in particular at about 0° C. to about 40° C.

Depending on the selection of crystallisation conditions, it may be advantageous to seed the crystallisation solution with a suitable crystallisation nucleus, for example a crystal of the compound preferably to be crystallised.

To carry out the method according to the invention the procedure is advantageously that a solution of the material mixture to be separated is heated in the selected solvent or solvent mixture, optionally with stirring, for example, as a function of the selected solvent or solvent mixture, to temperatures of about 40° C. to about 60° C., and then cooled slowly, i.e. over a time period of about 0.5 h to about 20 h to a temperature, at which the selective crystallisation of the coenzyme Q₁₀ starts (about 0-20° C.). If desired, the crystallisation can be completed by further lowering of the temperature.

As an alternative or in addition to this, it is also possible to provide a solution as described above of the material mixture to be separated in a suitable solvent or solvent mixture and to trigger the preferred selective crystallisation according to the invention by adding a further solvent or solvent mixture. In this case, inter alia both the crystallisation temperature and the manner of addition may be varied.

By means of the method according to the invention it is possible to provide coenzyme Q₁₀ in pure or enriched form, i.e. as a function of the purity or the content of coenzyme Q₁₀ of the starting material mixture, with a content of at least 70% by weight, preferably from about 80 to about 100% by weight, in particular from about 90 to about 99.5% by weight, particularly preferably from about 95 to about 99.5% by weight and most preferably from about 98 to about 99.5% by weight.

Furthermore, the separation method according to the invention may also be carried out by crystallisation from a melt of a material mixture containing coenzyme Q₁₀ of formula (I) and the compound of formula (II). Melt crystallisations of this type with the at least substantial absence of solvents are known to the person skilled in the art per se and described comprehensively, for example in G. F. Arkenbout, Melt Crystallisation Technology, Lancaster/PA, Technomic Publ. Co., 1995. In this case, both static and dynamic methods of suspension or layer crystallisation may be carried out according to the invention.

Analysis of the mixtures of compounds of formulas (I) and (II), mentioned as starting materials or as products of the method according to the invention is possible only with a large outlay for apparatus because of the large chemical and physical similarity of the molecules, which differ only by the arrangement of a few of the 50 carbon atoms of the side chain. Suitable methods for the analysis of similar material mixtures containing coenzyme Q₁₀ are described in USP 27, Official Monographs, page 2039 and in European Pharmacopoeia 5.0, page 2657.

A further embodiment of the method according to the invention relates to the production of pure or enriched coenzyme Q₁₀ by separating material mixtures containing coenzyme Q₁₀ and the compound of formula (II) by means of chromatographic methods, preferably on a preparative scale, in particular methods of normal-phase and reversed-phase chromatography being considered. In this case, the methods for normal-phase chromatography are to be regarded as preferred according to the invention.

A separation on a preparative scale is to be understood as one in which, in contrast to analytical separations, the fractions obtained are collected and isolated in a suitable manner, so they are available for further conversions or for use. In this case, separations are interesting in particular, in which substance quantities can be implemented in the range of above about 1 g through to the production scale. The method according to the invention for producing pure or enriched coenzyme Q₁₀ is accordingly in general, as well as with regard to said embodiments, a method for isolating said material in the pure or enriched form, preferably on a preparative or industrial scale and differs therefore from analytical methods, in which the smallest material quantities are separated but not isolated.

Methods for chromatographic purification of crude products or for separating material mixtures are known to the person skilled in the art and described comprehensively in Preparative Chromatography of Fine Chemicals and Pharmaceutical Agents, edited by Henner Schmidt-Taub, Wiley-VCH, 2005.

The chromatographic separation methods according to the invention, can be carried out at normal pressure or at elevated pressure. The separation according to the invention is preferably carried out at a pressure of 1 bar (absolute, i.e. without excess pressure) to 100 bar (abs.), particularly preferably of about 5 bar (abs.) up to about 80 bar (abs.).

The chromatography can be carried out in a temperature range of about 15 to about 80° C., i.e. the columns and the solvent are advantageously kept in the temperature range of about 15 to about 80° C., preferably at about 20 to about 40° C., particularly preferably at room temperature, i.e. at about 20 to about 25° C.

Suitable for carrying out the separation according to the invention by normal-phase chromatography are conventional materials suitable for application as stationary phases, such as, for example, silica gel (SiO₂) or aluminium oxide (Al₂O₃), preferably silica gel. The particle size can, in this case, be selected as a function of the selected mobile phase, or the respective separation problem or the sample volume to be separated within a broad range, but is generally about 5 μm to about 200 μm, preferably about 15 to about 100 μm.

In the scope of the separation method according to the invention, preferred separation materials are, for example, those with the designation silica gel 60 or silica gel 100 (Merck KgaA), LiChroprep® (Merck KGaA), for example LiChroprep® Si, LiChroprep® RP-2, LiChroprep® RP-8, LiChroprep® RP-18, LiChroprep® CN, LiChroprep® Diol, LiChroprep® NH2 (in each case Merck KGaA) or LiChrosper® (Merck KGaA), for example LiChrosper® Si, LiChrosper® CN, LiChrosper® NH2, LiChrosper® Diol (Merck KGaA) and LiChrosper® RP, as well as further materials known to the person skilled in the art as comparable. Particularly preferred in the scope of the present separation method are LiChroprep Si 60 and silica gel 60.

Suitable as the mobile phase in the scope of the preferred separation according to the invention by normal-phase chromatography are organic solvents or mixtures of various organic solvents, in which the isomers to be separated of formulas (I) or (II) or the optionally still present further components or impurities are adequately soluble. Mentioned by way of example as suitable solvents are the solvents listed above for carrying out the crystallisation according to the invention. Preferred amongst them are the hydrocarbons such as, for example, petrol ether, pentane, n-hexane, n-heptane, cyclohexane, preferably n-heptane and carbonyl compounds, such as, for example, acetone, diethyl ketone, methyl ethyl ketone, acetic acid ethyl ester or cyclohexanone, preferably acetic acid ethyl ester, as well as cyclic or acyclic ethers such as, for example, diethyl ether, tetrahydrofurane, dioxane or methyl-tert.-butyl ether.

Said solvents may, if used in the form of mixtures, be mixed with one another in any ratio. In this case, the selected mixing ratios may be kept constant in the course of the separation (isocratic mode of operation) or changed continuously or gradually (gradient mode of operation). Solvent mixtures preferred as the mobile phase according to the invention consist of acetic acid ethyl ester and a hydrocarbon, preferably n-heptane or n-hexane. In the isocratic mode of operation, the proportion of acetic acid ethyl ester in these solvent mixtures is preferably up to about 10% by volume, particularly preferably up to about 5% and quite particularly preferably about 0.5 to about 5% by volume.

In addition, the pH of the mobile phase may be varied by addition of acids or bases. For example, the pH of the respectively used mobile phase can be adjusted by the addition of acids, for example trifluoroacetic acid, to a pH of less than 7. When using the aforementioned solvent mixtures of hydrocarbons, preferably n-heptane or n-heptane and acetic acid ethyl ester, trifluoroacetic acid, generally in a quantity of up to about 1% by volume, preferably about 0.05 to about 1% by volume is generally advantageously added, for example.

The chromatography may be carried out discontinuously, i.e. as batch chromatography or else continuously. In the scope of a preferred embodiment of the method according to the invention, under suitable separation conditions, a continuous separation, which is particularly advantageous for applications on a preparative or industrial scale, can also be carried out under so-called simulated moving bed (SMB) conditions, such as described, for example, in Preparative Chromatography of Fine Chemicals and Pharmaceutical Agents, edited by Henner Schmidt-Taub, Wiley-VCH, 2005 or in Strube et al., Org. Proc. Res. Dev. 2 (5), 305-319, 1998. In SMB chromatography, the mobile and stationary phase are guided in simulated counter flow. The advantage is the lower use of solvents and stationary phase and the high purity of the product and recovery rate. In the case of separation of the mixture from coenzyme Q₁₀ and the isomeric formula (II) by SMB chromatography, it is advantageous to remove, prior to the actual chromatography, more polar components by a filtration over silica gel or by extraction from the crude product mixture.

The material mixture to be separated according to the invention by SMB chromatography is generally used in the form of a solution advantageously in the solvent or solvent mixture selected as the mobile phase. The concentration of this solution of the starting material mixture (feed) to be separated for the SMB chromatography can be selected from about 10 g/l up to the solubility limit of the starting material in the respective solvent or solvent mixture; it is preferably about 100 to about 120 g/l (based on the material mixture).

The mobile phase is generally moved through the column in the course of the SMB chromatography according to the invention at an empty tube speed of about 100 to 2,000 cm/h, preferably of about 800 to 1,200 cm/h. The pressure may be about 1 bar, i.e. without excess pressure, up to about 100 bar, preferably 35 to 60 bar (abs.). The solvent mixture is preferably a mixture of acetic acid ethyl ester and n-heptane or n-hexane with a proportion of up to 5% by volume of acetic ester. Quite particularly preferably, the ratio of acetic acid ester, based on the volume, to n-heptane or n-hexane is 98:2.

The method mentioned above for chromatographic separation of the isomeric compounds (I) and (II) can also be combined in the course of a preferred embodiment of the method according to the invention with the aforementioned crystallisation methods. Thus, it may be advantageous, for example following a chromatographic separation or an enrichment as described above of the desired isomer of formula (I), to subject the enriched product thus obtained to a crystallisation or a sequence of crystallisations as described above.

In this case, the upstream chromatographic separation or enrichment can also be carried out, for example, in the form of so-called flash chromatography or column filtration, in which the isomer mixture can firstly be partially or completely freed from further optionally present impurities, reagents or by-products and a depletion of the isomer of formula (II) already optionally takes place.

For example, in a first chromatographic stage to be designated pre-purification, a crude product mixture of the chemical synthesis of coenzyme Q₁₀ with a typical content of coenzyme Q₁₀ of formula (I) of typically about 60 to about 70% by weight can be used. A material mixture with a content of about 80 to about 95% by weight, often with about 85 to about 95% by weight coenzyme Q₁₀ of formula (I) is generally obtained therefrom, for example by normal-phase flash chromatography on silica gel with mixtures of acetic ester and a hydrocarbon. This enriched product mixture can be further purified then by crystallisation to be carried out according to the invention or a sequence of crystallisations.

The present invention accordingly also relates to a method for producing pure or enriched coenzyme Q₁₀ of formula (I)

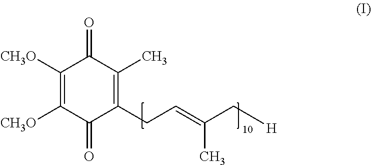

by separating material mixtures containing coenzyme Q₁₀ and the compound of formula (II)

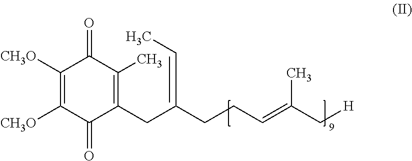

wherein, for separation, at least one chromatography and at least one crystallisation is carried out.

According to the invention, said separation methods are expediently carried out one after the other, the enriched product mixture obtained in the first separation step being supplied to the second separation step. Chromatography is preferably firstly carried out as a pre-purification and the enriched or pre-purified product mixture thus obtained is then subjected to a crystallisation as described above. If desired, said separation steps can also be carried out several times, preferably 2 or 3 times one after the other if no satisfactory enrichment was achieved by carrying out the respective separation step once.

When the individual separation steps are carried out repeatedly, regardless of whether these are carried out in the form of combinations of various separation methods or as a repetition of the same separation method, the separation conditions, for example the selection of solvents, stationary separation phases or other parameters, such as pressure or temperature, at which the individual separation steps are carried out, may be varied in each case or kept constant.

Moreover, said mixtures can also be separated or enriched in the manner according to the invention in that they are brought into contact with a medium which has groups, structures or functionalities, which are in a position to form a selective interaction preferably with one or two compounds of formulas (I) and (II), as they are used for example in affinity chromatography.

To achieve the desired results, it may be advantageous to carry out said preferred separation methods repeatedly one after the other, generally 2 to 5 times, preferably 2 to 3 times.

The efficiency of the methods according to the invention is surprising, as the two constitutional isomeric compounds of formulas (1) and (11) to be separated only differ in the arrangement of two of the carbon atoms of the polyisoprenoid side chain comprising a total of 50 carbon atoms. The person skilled in the art would therefore not have considered the possibility of separation according to the invention of said compounds in the manners described above.

The method according to the invention therefore opens up the possibility of providing isomer-pure or isomer-enriched coenzyme Q₁₀, which is suitable for use or administration to humans and animals. This type of material would not have been accessible otherwise by the convergent synthesis methods described in the introduction by transition metal-catalysed coupling of two structural synthesis elements.

### EXAMPLES

The following examples are used to describe the invention, without limiting them in any way. For analysis of said material mixtures, the above-mentioned methods according to USP 27 were used:

### Example 1

2.43 g of a mixture purified by column chromatography which consisted of 91.28% by weight coenzyme Q₁₀ and its isomer of formula (II) in the relative ratio 91.3 to 8.7, was dissolved in 50 ml ethanol, the solution heated with stirring to 50° C. and then cooled within 2 h to room temperature. The solution was then cooled to 0° C. and the crystals produced filtered off, rewashed with cooled ethanol and dried in a vacuum drying cabinet at 40° C. 2.01 g of a yellow solid was obtained, 98.86% by weight of which consisted of coenzyme Q₁₀ and the isomer of formula (II) in the relative ratio of 96.7 to 3.3.

### Example 2

1.32 g of the product obtained in Example 1 was dissolved in 25 ml ethanol, the solution heated with stirring to 50° C. and then cooled within 2 h to room temperature. The solution was then cooled to 0° C. and the crystals produced filtered off, rewashed with cooled ethanol and dried in a vacuum drying cabinet at 40° C. 1.28 g of a yellow solid was obtained, 96.9% by weight of which consisted of coenzyme Q₁₀ and the isomer of formula (II) in the relative ratio of 98.7 to 1.2.

### Example 3

45.6 g of a material mixture, 55.2% by weight of which consisted of coenzyme Q₁₀ and its isomer of formula (II), the compounds to be separated of formulas (I) and (II) being present in a relative ratio of 98.8 to 1.2 (HPLC surface %), was chromatographed over a pressure column (diameter: 8 cm, length: 50 cm, filled with silica gel, 0.04-0.063 mm). A mixture of hexane and acetic acid ethyl ester was used, the proportion of acetic ester being increased during the chromatography from 2 to 4% by volume. After removal of the solvent, 23.9 g of a mixture was obtained, of which 94.8% by weight consisted of coenzyme Q₁₀ and its isomer of formula (II) and the relative ratio thereof was 99.1:0.9 (HPLC surface %).

The mixture thus obtained was dissolved at 60° C. in 300 ml ethanol. The solution was then cooled at a rate of 5 K/h to 10° C. The orange solid precipitating in this case was sucked off, washed with 40 ml ethanol and dried in a vacuum drying cabinet at room temperature. 21.5 g of a solid was obtained, of which 97.7% by weight consisted of coenzyme Q₁₀ and its isomer of formula (II) and the relative ratio thereof was 99.7:0.3 (HPLC surface %).

### Example 4

15.6 g of a material mixture, of which 94.6% by weight consisted of coenzyme Q₁₀ and its isomer of formula (II), the compounds to be separated of formulas (I) and (II) being present in a relative ratio of 91.8 to 8.2 (HPLC surface %), was suspended in 80 ml ethanol and heated to 45° C. A further 300 ml ethanol was then added and after 30 min stirring, cooling took place at a rate of 5 K/h to 10° C. After 2 h stirring at 10° C., the solid was filtered off and washed with 20 ml cold ethanol. After drying, 12.7 g of a mixture was obtained, of which 100% by weight consisted of coenzyme Q₁₀ and its isomer of formula (II) and the relative ratio thereof was 97.6:2.4 (HPLC surface %).

The solid thus obtained was taken up in 190 ml ethanol and dissolved at 55° C. Stirring then took place for 2 h at 45° C. and cooling then took place at a rate of 5 K/h to 10° C. After stirring overnight at 10° C., the solid was filtered off, washed with 20 ml cold ethanol and dried. 11.9 g of a mixture was obtained, of which 100% by weight consisted of coenzyme Q₁₀ and its isomer of formula (II) and the relative ratio thereof was 99.1:0.9 (HPLC surface %).

The solid thus obtained was then again taken up in 200 ml ethanol and crystallised as before. 11.2 g of a mixture was obtained, 100% by weight of which consisted of coenzyme Q₁₀ and its isomer of formula (II) and the relative ratio of which was 99.6:0.4 (HPLC surface %).

### Example 5

23.8 g of a crude mixture containing 51.7% by weight of a mixture of coenzyme Q₁₀ of formula (I) and the compound of formula (II) in the relative ratio 97.9:2.1 (HPLC surface %) was filtered over a suction filter (4.5 cm height) filled with 250 g silica gel. At the beginning, elution took place with n-hexane and in the course of the filtration up to 10% by volume diethyl ether was added slowly. 12.3 g of a mixture was obtained, of which 87.7% by weight consisted of coenzyme Q₁₀ and its isomer of formula (II) and the relative ratio thereof was 98.5:1.5 (HPLC surface %).

8.8 g of the solid thus obtained was heated in 200 ml ethanol to 55° C. and a further 100 ml ethanol added. The solution was cooled at a rate of 5 K/h to 10° C., seeding taking place at 45° C. with 2 mg pure coenzyme Q₁₀. The solid was sucked off and washed with 20 ml ethanol. 7.4 g solid was obtained, consisting of 95.6% by weight coenzyme Q₁₀ and its isomer of formula (II), the relative ratio of which was 99.2:0.8 (HPLC surface %).

### Example 6

103.4 g of a material mixture, containing 60.9% by weight coenzyme Q₁₀ and its isomer of formula (II) in the relative ratio of 99.1:0.9 were chromatographed by means of MPLC (Medium pressure liquid chromatography) at a pressure of 8-10 bar with a solvent flow of 100 to 120 ml/min (column: diameter 10 cm, h=45 cm, filled with silica gel (LiChroprep® Si 60 15-25 μm, Merck). The chromatography was started with pure hexane. During the chromatography, acetic acid ethyl ester was added up to a proportion of 6% by volume (gradient mode of operation). 59.7 g of a product was obtained, of which 97.5% by weight consisted of coenzyme Q₁₀ and its isomer of formula (II) and the relative ratio thereof was 99.3:0.7 (HPLC surface %).

44 g of the solid thus obtained was dissolved at 60° C. in 500 ml ethanol. Cooling then took place at a rate of 10 K/h to 10° C. The cloudy solution was then seeded with a spatula tip of coenzyme Q₁₀ at 40° C., whereupon the solid formation started. The solid was filtered off at 10° C., washed with 95 ml ethanol and dried at 20 mbar at room temperature. 39.7 g of a solid was obtained, of which 95.7% by weight consisted of coenzyme Q₁₀ and its isomer of formula (II) and the relative ratio thereof was 99.6:0.4 (HPLC surface %).

### Example 7

60.3 g of a material mixture, of which 77.6% by weight consisted of coenzyme Q₁₀ and its isomer of formula (II) and the relative ratio thereof was 98:2 (HPLC surface %), was dissolved at 50° C. in 180 ml of a solvent mixture of ethanol and toluene in a volume ratio of 9 to 1. The mixture was then cooled at a rate of 5 K/h to 10° C. The solid produced was sucked off at 10° C. and rewashed with 30 ml cold ethanol/toluene. After drying, 9.5 g of a mixture was obtained, of which 84.9% by weight consisted of coenzyme Q₁₀ and its isomer of formula (II) and the relative ratio thereof was 97.9:2.1 (HPLC surface %).

### Example 8

30 g of a material mixture, of which 71.7% by weight consisted of coenzyme Q₁₀ and its isomer of formula (II) and the relative ratio of which was 92.1:7.9 (HPLC surface %), was dissolved at 50° C. in 180 ml of a solvent mixture of ethanol and acetone in a volume ratio of 7 to 3. The solution was then cooled to 30° C. and after seeding cooled further at 5 K/h to 10° C. The solid produced was sucked off and rewashed with 30 ml of the ethanol/acetone mixture. After drying, 22.8 g of a mixture was obtained, of which 80.3% by weight consisted of coenzyme Q₁₀ and its isomer of formula (II) and the relative ratio thereof was 96.5:3.5 (HPLC surface %).

### Example 9

To separate a mixture of coenzyme Q₁₀ and the isomer of formula (II) in the ratio of 94 to 6, using n-heptane as the main component of the solvent and using the following stationary phases the following were investigated: LiChroprep® RP-2, 25-40 μm; LiChroprep® Si 60, 5-20 μm; LiChroprep® Si 60, 12 μm; LiChroprep® CN, 25-40 μm; LiChrospher® 100 CN, 10 μm; LiChrospher® 100 NH2, 15 μm; LiChrospher® 100 Diol, 10 μm.

The best separation performance was achieved with the LiChroprep® Si 60-column as the stationary phase. Table 1 summarises the solvent compositions used in this system and the separation results achieved:

TABLE 1

| | | | | k′-value | |
|---|---|---|---|---|---|
| | | | k′-value | (Coenzyme | |
| Solvent | Ratio | Temperature | (Isomer) | Q₁₀) | alpha |
| Heptane/MtBE | 95/5 | RT | 9.05 | 10.02 | 1.11 |
| Heptane/MtBE | 96/4 | RT | 10.36 | 11.65 | 1.12 |
| Heptane/MtBE | 97/3 | RT | 10.89 | 11.87 | 1.09 |
| Heptane/EtAc | 98/2 | RT | 23.45 | 25.58 | 1.09 |
| Heptane/EtAc | 98/2 | 15° C. | 23.65 | 25.46 | 1.08 |
| Heptane/EtAc | 98/2 | 15° C. | 23.48 | 25.39 | 1.08 |
| Heptane/EtAc | 98/2 | RT | 21.06 | 23.08 | 1.10 |
| Heptane/EtAc | 98/2 | 35° C. | 20.79 | 22.95 | 1.10 |
| Heptane/EtAc | 98/2 | 45° C. | 20.37 | 22.51 | 1.11 |
| Heptane/EtAc | 98/2 | 45° C. | 18.45 | 20.51 | 1.11 |
| Heptane/EtAc | 98/2 | 55° C. | 16.9 | 18.78 | 1.11 |
| Heptane/EtAc | 97/3 | 15° C. | 9.42 | 10.49 | 1.11 |
| Heptane/EtAc* | 98/2 | RT | | 13.2 | |
| Heptane/EtAc** | 98/2 | RT | 9.48 | 10.81 | 1.14 |
| Heptane/EtAc** | 98/2 | 15° C. | 9.8 | 11.25 | 1.15 |
| Heptane/EtAc** | 99/1 | RT | 19.85 | 22.74 | 1.15 |
| Methyl | 98/2 | RT | | 11.46 | |
| cyclohexane/EtAc | | | | | |
| Methyl | 100 | RT | | No | |
| cyclohexane | | | | separation | |
| Methyl | 99/1 | RT | | No | |
| cyclohexane/EtAc | | | | separation | |

*Addition of 0.1% by volume triethylamine
**Addition of 0.1% by volume trifluoroacetic acid

### Abbreviations:

RT: room temperature; MtBE: methyl-tert.butyl ether; EtOAc: acetic acid ethyl ester k′-value: retention factor
alpha: selectivity (k′-value coenzyme Q₁₀/k′-value (isomer)

The best results were achieved in the solvent heptane/acetic acid 98/2 with the addition of 0.1% trifluoroacetic acid. The precise separation conditions are given in Table 2; the eluents A and B were mixed according to the gradients given in Table 3:

TABLE 2

| | |
|---|---|
| Column: | LiChroprep Si 60 (5-20 μm) |
| Eluent: | A: 98/2 n-heptane/ethyl acetate + 0.1% TFE |
| | B: ethyl acetate |
| Empty tube speed | 1000 cm/h |
| Column temperature: | 22° C. |
| Detection UV VIS: | 270 nm |
| Pressure: | 35 bar |
| Sample solvent: | 98/2 n-heptane/ethyl acetate + 0.1% TFE |
| Sample concentration: | 10 g/l (max. solubility limit) |

TABLE 3

| Time | A | B | Flow rate |
|---|---|---|---|
| [min.] | [Vol.-%] | [Vol.-%] | [ml/min.] |
| 0 | 100 | 0 | 2 |
| 10 | 100 | 0 | 2 |
| 20 | 0 | 100 | 2 |
| 25 | 0 | 100 | 2 |
| 25.1 | 100 | 0 | 2 |
| 30 | 100 | 0 | 2 |

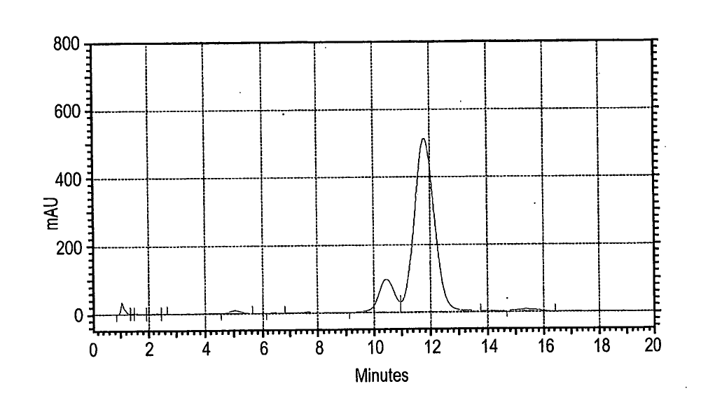

FIG. 1 shows a typical chromatogram for a discontinuous separation according to Example 9.

1. Method for producing pure or enriched coenzyme Q₁₀ of formula (I) 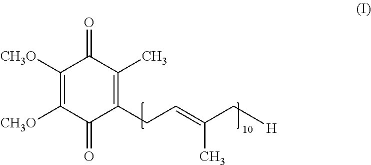
by separating material mixtures containing coenzyme Q₁₀ and the compound of formula (II)

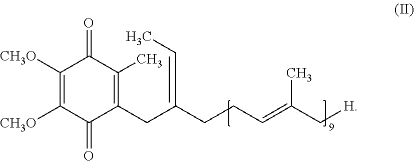

2. Method according to claim 1, characterised in that, for separation, a selective crystallisation of coenzyme Q₁₀ is carried out from a solution or a melt of material mixtures containing coenzyme Q₁₀ and a compound of formula (II).

3. Method according to claim 2, characterised in that the crystallisation is carried out from solutions of said material mixtures containing ethanol and/or acetone as the solvent.

4. Method according to claim 2 or 3, characterised in that the crystallisation is carried out from a solvent or solvent mixture, of which 70 to 100% by volume consists of ethanol.

5. Method according to any one of claims 2 to 4, characterised in that the crystallisation is carried out at temperatures in the range of −20° C. to 80° C.

6. Method according to any one of claims 2 to 5, characterised in that solutions are used which, based on the total solution, contain 1 to 35% by weight of said material mixture.

7. Method according to any one of claims 1 to 6, characterised in that material mixtures are used, in which coenzyme Q₁₀ of formula (I) and the compound of formula (II) are present in the molar ratio of 85 to 15 up to 99.7 to 0.3.

8. Method according to claim 1, characterised in that chromatography is carried out for separation.

9. Method according to claim 8, characterised in that at least one chromatography and at least one crystallisation is carried out for separation.

10. Method according to claim 8 or 9, characterised in that chromatography is carried out on a preparative scale.

11. Method according to any one of claims 8 to 10, characterised in that normal-phase chromatography is carried out using silica gel as the stationary phase.

12. Method according to any one of claims 8 to 11, characterised in that the chromatography is carried out at a pressure of 1 to 80 bar.

13. Method according to any one of claims 8 to 12, characterised in that the chromatography is carried out with a solvent mixture of acetic acid ethyl ester and n-heptane or acetic acid ethyl ester and n-hexane, the proportion of acetic acid ethyl ester being up to 5% by volume in each case.

14. Method according to claim 13, characterised in that trifluoroacetic acid in a quantity of up to 5% by volume is added to the solvent mixture of acetic acid ethyl ester and n-hexane or n-heptane.

15. Method according to any one of claims 8 to 14, characterised in that the chromatography is carried out at a temperature range from 15 to 60° C., preferably at a temperature range of 20 to 25° C.

16. Method according to claim 1, characterised in that affinity chromatography is carried out for separation.
